(19) United States
(12) Patent Application Publication
(10) Pub. No.: US 2025/0381417 A1
(43) Pub. Date: Dec. 18, 2025

(54) Methods and Systems for Optimizing Radiation Therapy Treatment Planning Including Modulator Configuration

(71) Applicant: Emory University, Atlanta, GA (US)

(72) Inventors: Jun Zhou, Atlanta, GA (US); Xiaofeng Yang, Atlanta, GA (US); Chaoqiong Ma, Atlanta, GA (US); Ahmal Jawad Zafar, Atlanta, GA (US)

(21) Appl. No.: 19/236,861

(22) Filed: Jun. 12, 2025

Related U.S. Application Data
(60) Provisional application No. 63/659,291, filed on Jun. 12, 2024.

(51) Int. Cl. A61N 5/10; G16H 20/40

(52) U.S. Cl. CPC A61N 5/1031; A61N 5/1042; G16H 20/40

(57) ABSTRACT

The systems and devices can optimize a modularized beam modulator configuration using a library of components, while optionally simultaneously optimize the radiation delivery parameters. In one implementation, the method may include applying one or more optimization procedures to one or more candidate radiation treatment plans for radiation treatment in a patient according to one or more plan optimization objectives associated with one or more cost functions to generate a final radiation treatment plan. In some examples, each radiation treatment plan may include therapy parameters and a beam modulator configuration of one or more geometric components from a library storing a plurality of the modular components. In some examples, one or more optimization procedures may be applied to the beam modulator configuration of each candidate radiation treatment plan to generate a final beam modulator configuration. The final treatment plan may include the final beam modulator configuration.

### CROSS-REFERENCE TO RELATED APPLICATIONS

This application claims the benefit of U.S. Provisional Application No. 63/659,291 filed Jun. 12, 2024. The entirety of this application is hereby incorporated by reference for all purposes.

### BACKGROUND

Current challenges to proton therapy (i.e., IMPT) planning generally include particle range uncertainties and high sensitivity to anatomical changes. To achieve the high dose rate in a modern proton/ion therapy device and address these challenges, a 3D printed, patient-specific beam modulator, such as a ridge filter, has been typically used to create a spread-out Bragg peak (SOBP) without switching the energy layers. This approach can result in slow fabrication of the modulator, high costs, and inability to adjust during treatment.

### SUMMARY

Thus, there is a need for techniques that can efficiently and accurately provide ion radiation therapy, while improving treatment results.

Techniques disclosed herein relate generally to systems and devices that can optimize a modularized beam modulator configuration using a library of components, while optionally simultaneously optimize the radiation delivery parameters. This can allow for beam modulators and radiation delivery parameters that can be easily adjusted to adapt to patient's anatomy changes for online/offline adaptive FLASH therapy. Thus, the techniques can accelerate production and reduce costs associated with fabricating beam modulators while allowing for easy adjustments during treatment and improving treatment results.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, the emphasis being placed upon illustrating the principles of the disclosure.

FIG. 1 illustrates an example of a system environment for optimizing a radiation treatment plan according to some embodiments.

FIG. 2 is a flow chart illustrating a process of optimizing a radiation treatment plan according to some embodiments.

FIG. 3A is an illustrative example of generating a graphical representation of a filter configuration according to some embodiments. FIG. 3B is an illustrative example of a matrix generated using the graphical representation.

FIG. 4 is an illustrative example of generating a graphical representation of proton intensity according to some embodiments.

FIG. 5 shows an illustrative example of a workflow for optimizing a radiation treatment plan according to embodiments.

FIG. 6 is a simplified block diagram of an example of a computing system for implementing certain embodiments disclosed herein.

### DESCRIPTION OF THE EMBODIMENTS

In the following description and Appendices, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

This disclosure relates to methods and systems that can jointly optimize a multi-energy particle therapy plan, for example, generated using a conventional treatment planning system. For example, the methods and systems can optimize treatment plan parameters, such as physical beam modulator configuration and radiation therapy parameters (e.g., proton spot intensity), of a treatment plan for particle therapy, particularly suited for ultra-fast beam delivery applications, such as proton FLASH therapy. The methods and systems use an optimization procedure that combines Graph Neural Networks (GNNs) with combinatorial optimization, such as Quadratic Unconstrained Binary Optimization (QUBO) matrices, to iteratively refine both parameters based on a cost function reflecting clinical objectives. In some examples, each modulator component (e.g., step) and proton spot can be represented as a graph node with binary actions indicating whether to retain, increase, or decrease its contribution. In some examples, QUBO matrices can quantify the impact of individual and combined changes on treatment quality, guiding convergence to an optimal configuration and/or proton intensity. In some examples, the optimized treatment plan generated by one or more optimization procedure can result in a plan that defines discrete spot locations arranged in a lattice pattern, fixed set of allowable proton energies derived from the model, and preliminary intensity values for each spot.

The disclosed methods and systems also use a predefined library of modular filter steps, enabling fast assembly, reuse of physical components, and easy adaptation to patient-specific anatomical changes or retreatment scenarios. The disclosed methods and systems can offer significant improvements in both planning efficiency and delivery accuracy over traditional manual or heuristic-based methods.

The disclosed methods and systems can be used to optimize a treatment plan (e.g., treatment plan parameters, such as therapy parameters and/or modulator configuration) for a radiation treatment therapy that can be used to treat cancer or other ailments in patients (e.g., human or animal). In some examples, radiation therapy may be provided by using particles, such as proton, other light ion therapies, other heavy ion therapies, among others, or any combination thereof. For example, the therapies may be single and/or multi-energy proton therapies.

Various embodiments are described herein, including systems, methods, devices, modules, models, algorithms, networks, structures, processes, computer-program products, and the like.

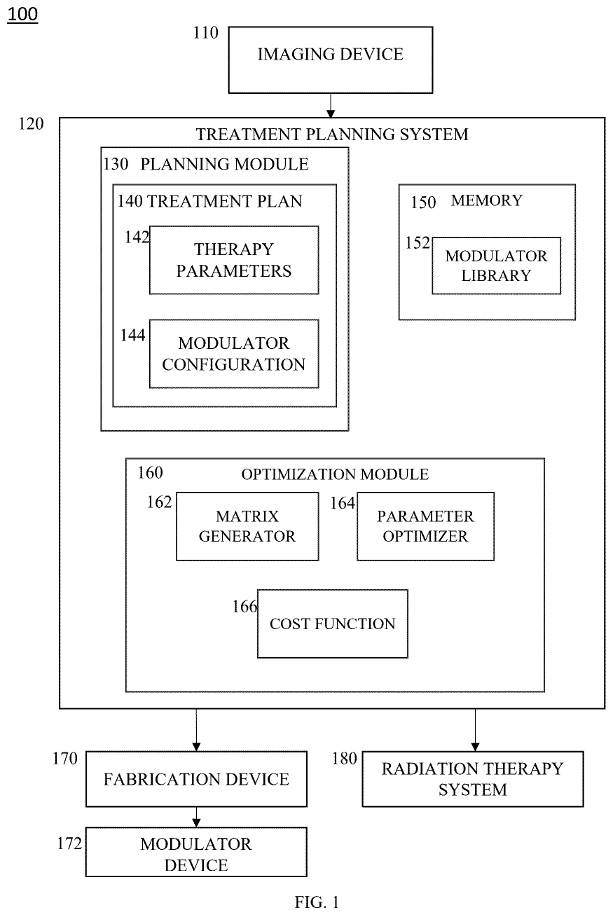

FIG. 1 depicts an example of a system environment 100 for generating an optimized radiation treatment plan (also referred to as “radiation therapy treatment plan” or “radiation therapy plan”) according to embodiments. The system environment 100 may include an imaging device 110, a treatment planning system 120 configured to generate a radiation treatment plan 140, a beam modulator fabricating device 170 configured to fabricate a modularized beam modulator device (also referred to as “beam modulator”) 172 according to the optimized (or “final”) treatment plan 140, and a radiation therapy system 180 configured to deliver the radiation therapy according to the optimized treatment plan 140 by controlling components such as, activate the gantry, the particle source, the accelerator, the magnets, particle beam, or the like.

These components may be in communication with each other via wired or wireless links. Other systems may also be used. It is also to be understood that the system environment 100 may omit any of the modules illustrated (e.g., the imaging device 110, the beam modulator construction device 170, and/or the radiation therapy system 180 and/or may include additional modules not shown.

In some examples, the treatment planning system 120 may be configured to generate, optimize, and evaluate candidate (proposed) treatment plans 140 and generate a final (optimized) treatment plan 140. Each treatment plan 140 may include treatment plan parameters. The treatment plan parameters may include but is not limited to therapy parameters 142, a beam modulator configuration 144, among others or any combination thereof.

In some examples, the therapy parameters 142 may include values associated with dose parameters that can affect dose and/or dose rate, as well as other parameters. The therapy parameters 142 may depend on the particle therapy to be delivered by the radiation therapy system 180. In some examples, for proton radiation therapy, the therapy parameters 142 may include but is not limited to beam shape (collimation); number and arrangement of spots for spot (pencil beam) scanning, and spot intensities (also referred to as “spot monitoring units” or “spot MUs”); beamlet (e.g., energy layer) weights and/or energies; beam/beamlet directions; prescribed dose and prescribed dose rate; a number of irradiations of a target volume; a duration of each of the irradiations (irradiation times); a dose deposited in each of the irradiations; among others; or any combination thereof.

In some examples, the beam modulator configuration 144 may relate to an arrangement of a number of energy degrading units formed by one or more modulation geometric components configured to spread energy that form a beam modulator device 172. In some examples, the beam modulator may be configured to produce a single-energy spread-out Bragg peaks (SOBP) along each pencil bean direction (PBD).

In some examples, the beam modulator device 172 may include an arrangement of one or more multi-layered or stacked energy degrading units. In some examples, the beam modulator device 172 may include but is not limited to a ridge filter device, such as a pin ridge filter, as shown and described in the figures. In this example, each energy-degrading unit may correspond to a pin. In other examples, the beam modulator device 172 may be a different beam modulator.

In some examples, each geometric component may correspond to a modular component stored in the library 152. In some examples, the one or more modular components stored in the library 152 may include one or more pins with predefined shape and size, one or more bars with predefined shape and size, other geometric shapes, among others. In some examples, the beam modulator configuration 144 and the resulting beam modulator device 172 may include one or more pins of different sizes, one or more bars of different sizes, among others, or any combination thereof. In some examples, as shown in the figures, the library 152 may store a number of different step or bar shapes. In some examples, each of the geometric components stored in the library 152 may correspond to a pre-fabricated module component available to be used to fabricate the beam modulator device 172.

In some examples, the library 152 may include components with different widths or weights (cross-sectional area of the step) and/or thicknesses (height). For example, the library 152 may include any number of geometric components with varying widths and/or heights. In some embodiments, the library 152 can include a plurality of components having varying width and/or height. For example, the components may vary in size incrementally. By way of example, if the library stores six components (e.g., six steps) that vary in width from 1 mm-6 mm, the first component may have a width of 1 mm, the second component may have a width of 2 mm, . . . , the sixth component may have a width of 6 mm. By way of another non-limiting example, examples of components that may be stored in the library are described in Ma et al. Streamlined pin-ridge-filter design for single-energy proton FLASH planning. Med Phys. 2024; 51:2955-2966, which is incorporated by reference in its entirety. In other examples, different sized and/or shaped components may be stored in the library.

In some embodiments, the beam modulator configuration 144 may be generated by translating therapy parameters associated with each treatment plan into a beam modulator configuration 144 using the library 152 for a single-energy radiation therapy plan, and the resulting beam modulator device 172, may be generated using the prefabricated module components corresponding to the arrangement of the geometric components of the (final) beam modulator configuration 144.

In some examples, the treatment planning module 130 may be configured to generate the one or more candidate treatment plans and a final treatment plan for a patient. For example, the treatment planning module 130 may be configured to generate the initial (also referred to as “first candidate”) treatment plan, such as an intermediate-modulated proton therapy (IMPT) plan, for the patient using the three-dimensional image data of the patient acquired by the imaging device 110. In some examples, the treatment planning module 130 may be configured to translate each treatment plan (e.g., the therapy parameters 142) into the modulator configuration 144 (which can be considered to be a part of “candidate” treatment plan (e.g., using the initial or optimized therapy parameters 142)). In some examples, the treatment planning module 130 may be configured to generate a plurality of candidate treatment plans in which one or more treatment plan parameters differ (e.g., different therapy parameter(s) 122 and/or modulator configurations 144).

By way of example, the optimization module 160 may be configured to optimize and evaluate each candidate treatment plan using one or more optimization procedures. Based on that evaluation, the treatment planning module 130 may generate the final treatment plan 140.

In some examples, the optimization module 160 may include one or more matrix generators 162, one or more parameter optimizers 164, and one or more cost functions 166 on which the candidate treatment plan(s) may be evaluated. In some examples, the optimization module 160 may be configured to perform one or more optimization procedures configured to optimize a treatment plan by optimizing the value of the one or more therapy parameters 142 (e.g., spot intensity), the geometric components (selected from the library 152) of the beam modulator configuration 144, among others, or a combination thereof.

In some examples, the one or more optimization procedures may include a first optimization procedure applied to the beam modulator configuration 144 and a second optimization procedure applied to the one or more therapy parameters 142. For example, if performed sequentially, the first optimization procedure may be iteratively applied to the beam modulator configuration 144, with the therapy parameter(s) 142 held constant, until the cost function for the beam modulator configuration 144 is satisfied; and the second optimization procedure may be applied to the therapy parameters 142 (updated based on the final beam modulator configuration 144 from the first optimization procedure) until the cost function for the therapy parameters 142 are satisfied. In other examples, the optimization procedures may be performed simultaneously. Each procedure may include one or more iterations.

In some examples, for each optimization procedure, the optimization module 160 may generate a graphical representation of one or more candidate parameters of the candidate treatment plan. For example, the graphical representation for the beam modulator configuration 144 may be generated by encoding each component of the beam modulator configuration 144 into a graphical representation so that each node of the graphical representation represents a respective component of the modulator configuration 144 with a binary action and one or more physical features associated with the respective component. For example, the one or more physical features associated with the node may include the size of the component (e.g., width and/or height), the physical relationship of the respective component position (e.g., position within the beam modulator) to other components of the beam modulator configuration 144, among other physical features, or any combination thereof.

For example, the binary action may be represented by a variable having value of 0 or 1. Each value of the binary variable (xᵢ) may dictate a predefined action with respect to the component. For example, if the value is 1, a change (increase or decrease size) is assigned to that component and if the value is 0, no change is assigned to that component. The predefined action associated with “1” (e.g., increase in one size increment or decrease in one size increment) may depend on the iteration number of the procedure. Before each iteration of the first optimization procedure, the binary node actions for each component (step) may be initialized to 0.

For example, the graphical representation for the therapy parameters 142, such as spot intensity, may be generated so that each node of the graphical representation represents a representative spot with a binary action and one or more physical features associated with the respective spot. For example, the one or more physical features associated with the node may include the current intensity and the physical relationship of the respective spot position to other spots, among other physical features, or any combination thereof.

Like the modulator configuration 144, the binary action may be represented by a variable having value of 0 or 1. Each value of the binary variable (xᵢ) may dictate a predefined action with respect to Monitor Units (intensity) of the spot. For example, a binary variable of “1” may indicate an increase or decrease in value of the MU and a binary variable of “0” may indicate no change in the MU of spot. For example, “1” may indicate a predefined increase in value in the value of MU. The predefined action associated with “1” (e.g., 10% increase, 5% increase, etc.) may depend on the iteration number of the optimization procedure. Before each iteration of the second optimization procedure, the binary node actions for each spot may be initialized to 0.

For each operation procedure, the optimization module 160 may include a matrix generator 162 configured to generate a matrix in which the parameters are mapped with respect to plan optimization objective(s) of the associated cost function. The matrix may be generated to capture both individual and pairwise interactions between candidate parameters (142 and/or 144) relative to the plan optimization objective(s). For example, the matrix may represent a combinatorial optimization problem. In some examples, the problem may be a quadratic unconstrained binary optimization (QUBO) problem and the matrix may be a QUBO matrix.

In some examples, the plan optimization objective(s) may be derived from clinical goals and/or treatment planning protocols stored in the memory 150 and/or associated with the radiation therapy treatment system 180. For example, the plan optimization objective(s) may include but are not limited to maximizing target dose coverage, minimizing dose to organs-at-risk (OARs), ensuring dose homogeneity, and maintaining dose fall-off outside the target.

In some examples, each cost function may be a mathematical formulation of parameters (parameters such as those mentioned above) that may have an effect on achieving the plan optimization objective(s). In some examples, the cost function(s) can be used to evaluate proposed radiation therapy treatment plans to determine whether or not the clinical goals that are specified for treatment of a patient defined by the plan optimization objective(s) are satisfied. By way of examples, non-limiting examples of cost functions can be found in Liu W et al. Robust optimization of intensity modulated proton therapy. Med Phys. 2012 February; 39 (2): 1079-91; and Webb S. Optimisation of conformal radiotherapy dose distributions by simulated annealing. Phys Med Biol. 1989 October; 34 (10): 1349-70; which are each incorporated by reference in their entirety.

For example, the matrix may include an element that represents a change in the respective cost value (e.g., representing a change in plan quality) associated with one or more predefined changes (e.g., dictated by the iteration number) for each candidate parameter. For example, for the modulator beam configuration 144, the matrix may be constructed such that each element of this matrix may represent a change in a quantitative measure corresponding to a predefined change (e.g., increase width and/or height by 1) in the component size of a unit of the modular ridge configuration.

For example, for therapy parameters 142, such as spot intensity, a matrix may be generated such that it includes an element corresponding to a predefined percentage change in the intensity of each individual spot or each pair of spots (e.g., dictated by the iteration number).

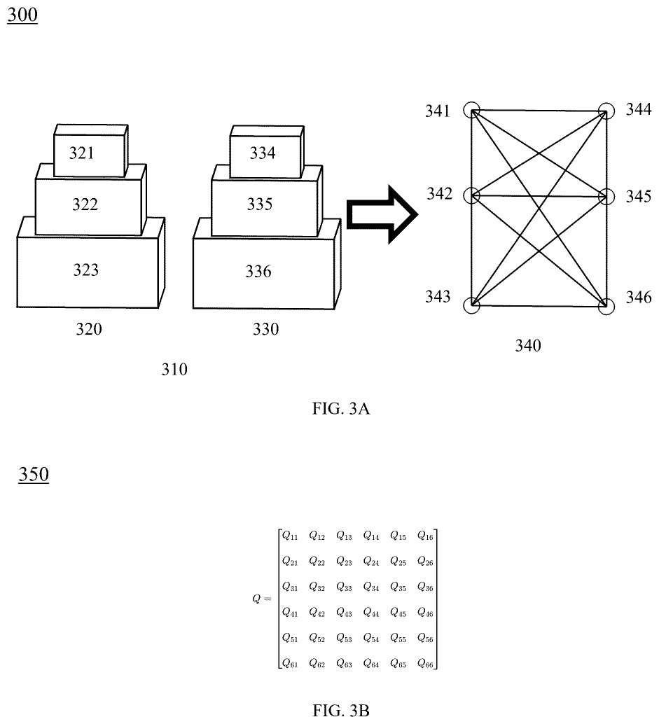

FIG. 3B shows an example of a 6×6 matrix 350. In each matrix, Qij can represent the interaction effect between each node i and node j, i.e., whether making both changes together is more (or less) beneficial than the sum of their individual impacts. Each binary variable xᵢ can represent a discrete decision. For example, if xᵢ=1, the binary action can indicate application of a specific change (e.g., increase and/or decrease intensity of spot i by a predefined amount; increase and/or decrease step size of the component at the given position); and if xᵢ=0, the binary action can indicate maintaining the current spot/component (i.e., no change).

In some examples, interaction effect between nodes may be quantified. The cost function value (Lₒ) (also referred to as baseline cost function) may first be determined for the baseline/initial treatment plan (no changes). In some examples, the different interaction terms may be determined.

For example, diagonal terms (Qᵢᵢ) may be determined. In this example, for each node xᵢ, the cost when that the single change is applied may be determined. By way of example, to compute the diagonal term for each element, a new function value (Lᵢ) may be determined by changing only spot or step i, keeping all others unchanged. The respective (diagonal term) element may be determined as follows: Qᵢᵢ=Lᵢ−Lₒ.

Next, off-diagonal Terms (Qᵢⱼ) be determined. In this example, for each pair (i, j), changes may be applied to both and a new function value (Lᵢⱼ) may be determined when both xᵢ=1 and xⱼ=1. The respective (non-diagonal term) element may be determined as follows: Qᵢⱼ=Lᵢⱼ−Lᵢ−Lⱼ+Lₒ.

These terms can quantify the interaction effect by indicating whether making both changes together is more (or less) beneficial than the sum of their individual impacts.

Next, a parameter optimizer 164 may be applied to each matrix to evaluate each action for each step. In some examples, the parameter optimizer 164 may be a neural network, such as a graphical neural network (e.g., a Graph Neural Network (GNN)), quantum annealing-based optimizer, simulated annealing-based optimizer, other optimization techniques and/or solvers, among others, or any combination thereof. In some examples, the GNN may be applied to each matrix to generate binary variables (corresponding to respective actions) that optimize the cost function based on the linear combinations of the elements within the matrix. The GNN may be configured to evaluate combinations of these binary variables to minimize the total cost, ultimately guiding the Graph Neural Network (GNN) toward an optimal configuration of the modulator. This way, the parameter optimizer 164 may act as a solver of the QUBO problem defined by the matrix. For example, the GNN may generate (predict) optimized variables for each matrix.

For example, for the therapy parameters 142, the GNN may generate a binary variable representing a change (no change or change (e.g., predefined increase or decrease)) in intensity for each individual spot or each pair of spots. By way of another example, for the modulator configuration 144, the GNN may generate a binary variable representing a change (no change or change (e.g., predefined increase or decrease)) in components of each unit included in the configuration.

In some examples, the GNN may be trained using matrices and graphical representations to estimate optimized parameters. In other examples, the parameter optimizer 164 may be a quantum computer or quantum processor configured to solve the QUBO problem.

The optimization module 160 may perform one or more iterations of the one or more optimization procedures until stopping criteria is met. For example, the stopping criteria may include but is not limited to the plan optimization objective(s) of the cost function(s) are satisfied (e.g., cost value is within a specified threshold), a maximum number of iterations of the optimization procedure(s) has been reached, among others, or any combination thereof. After which, the final treatment plan may optionally be transmitted to the beam modulator construction device 170 and/or the radiation therapy device 180.

In some examples, the system environment 100 may optionally include the beam modulator fabricating device 170 that can fabricate beam modulator device according to the beam modulator configuration 144 determined by the system 120. In some examples, the beam modulator fabricating device 170 may be a robotic system configured to build the beam modulator 172 using pre-fabricated module components corresponding to the components stored in the library 152 according to the beam modulator configuration 144 associated with the (final) treatment plan 140. In some examples, the system 100 may further include another device that uses optical imaging for quality control of the beam modulator.

In some examples, the radiation therapy system 180 may optionally apply particle therapy to a treatment target of the patient according to the generated final treatment plan (therapy parameters 142). The radiation therapy system 180 may be a treatment modality for providing radiation therapy treatment, such as intensity modulated radiation therapy (IMRT) or intensity modulated particle therapy (IMPT). For example, the radiation therapy system 180 may be configured to deliver particle therapy, such as proton FLASH radiotherapy, non-FLASH proton radiotherapy, other particle radiation systems (e.g., ion), among others, or any combination thereof. By way of example, the radiation therapy system 180 may be configured to form a proton source into a proton beam with a desired intensity, and energy, according to the therapy parameters 142 of the final treatment plan 140 which can be directed through the beam modulator fabricated based on the beam modulator configuration 144 of the final treatment plan 140 and ultimately into area volume within the patient's body (e.g., cancer (tumor)). Non-limiting exemplary devices that may be used to administer FLASH radiation are described in, for example, U.S. Pat. No. 9,855,445, which is incorporated by reference, and proton FLASH radiotherapy systems and devices by VARIAN MEDICAL SYSTEM, proton FLASH radiotherapy systems by ELEKTA, and the like.

The memory 150 may include electronic memory (e.g., solid state memory, SRAM (static random-access memory), DRAM (dynamic random-access memory), and/or the like). The memory 150 may include computer-readable instructions, data structures, program modules, and the like associated with the treatment planning system 120. In some examples, the treatment planning system 120 may be alternatively a part of the radiation therapy system 180.

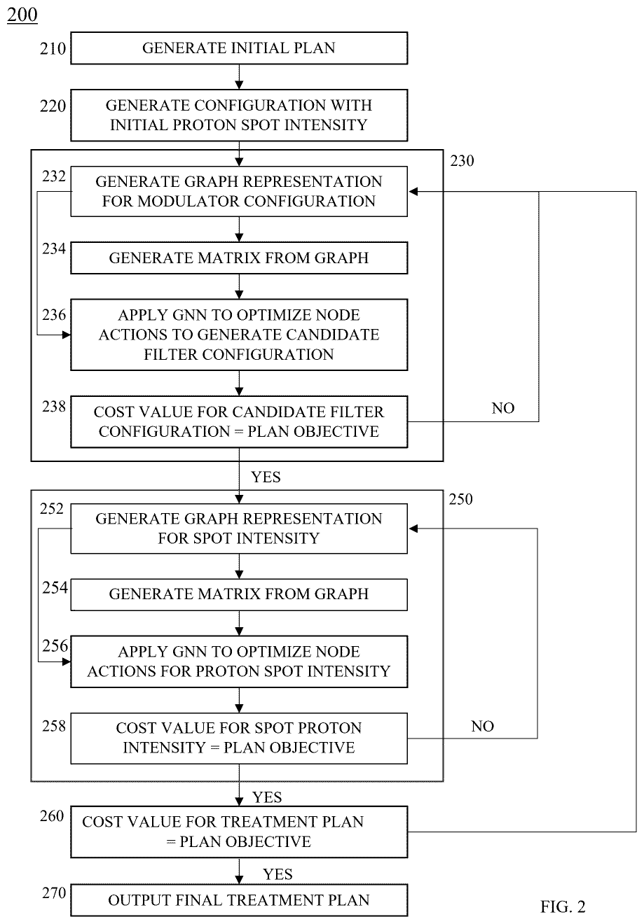

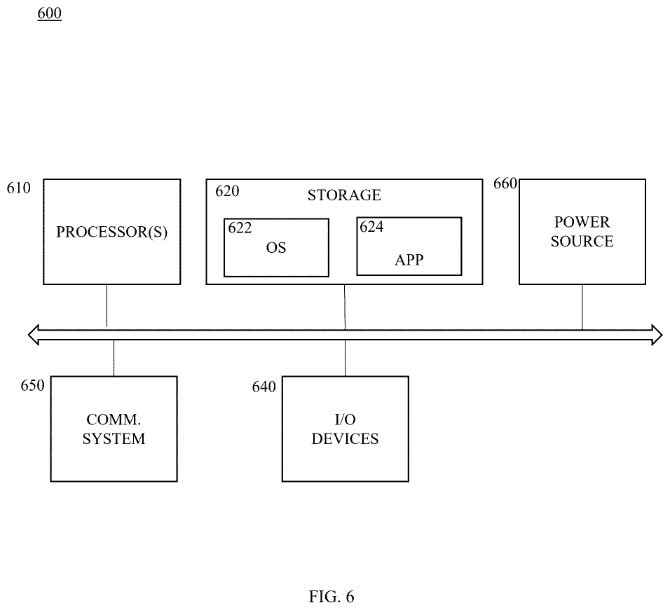

FIG. 2 shows an example of a flow diagram 200 illustrating a method for generating an optimized treatment plan that can include a modular beam modulator configuration and/or therapy parameters. Operations described in diagram 200 may be performed by a computing system, such as a computing system described below with respect to FIG. 6.

Although the flow diagram 200 may describe the operations as a sequential process, in various embodiments, some of the operations may be performed in parallel or concurrently. In addition, the order of the operations may be rearranged. An operation may have additional steps not shown in the figure. In some embodiments, some operations may be optional. Embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

Operations in flow diagram 200 may begin at block 210 when an initial treatment plan is generated using the medical image(s) of the patient and according to the clinical objectives for the treatment plan. In some examples, the initial treatment plan may be an intermediate intensity-modulated proton therapy (IMPT) plan. For example, the initial treatment plan may be generated using available methods.

Next, at block 220, the (initial) beam modulator configuration may be generated, for example, based on the initial plan generated at block 210 using the library 152. For example, the initial multi-energy treatment plan may be mapped into a single-energy delivery via the initial beam modulator configuration 144. By way of non-limiting example, the beam modulator configuration 144 may be generated according to method as described in Ma C et al. Streamlined pin-ridge-filter design for single-energy proton FLASH planning. Med Phys. 2024; 51:2955-2966, which is incorporated herein in its entirety. The beam modulator configuration 144 may be composed of modular geometric components selected from the library 152.

Next, in some examples, the first optimization procedure 230 to optimize the beam modulator configuration 144 may be performed. For example, at block 232, a graphical representation of the modulator configuration 144 may be generated from the initial beam modulator configuration. For example, each beam modulator configuration 144 may be encoded into a graphical representation that includes nodes representing individual components and their associated physical features. In the graphical representation, the binary actions associated with each node may be initialized to 0.

By way of example, FIG. 3A shows an illustrative example 300 of a graphical representation 340 of a beam modulator configuration 310. In this example, the configuration 310 includes two units 320 and 330. In this example, the first unit 320 may include components 321, 322 and 323 and the second unit 330 may include components 334, 335, and 336. As shown in the graphical representation 340, nodes 341-346 may correspond to components 321-323 and 334-336, respectively.

Next, at block 234, the matrix generator 162 may generate a matrix that includes elements representing a predefined change in the component size of a unit of the modulator configuration using the graphical representation and the optimization objectives derived from the clinical goals. In some examples, the predefined change may be based on the iteration number of the optimization procedure. By way of example, the predefined change for the first iteration of the (first) optimization procedure may be increase the width of the component (e.g., associated with binary value 1). The predefined change may differ between iterations.

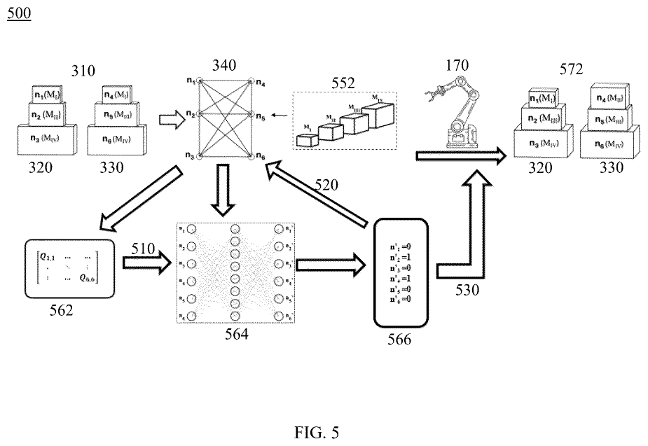

By way of example, for the example 300 shown in FIG. 3A, the matrix generator 162 may generate a 6×6 matrix 350 shown in FIG. 3B and FIG. 5, using the graphical representation 340. As shown, the 6×6 matrix 350 would capture both the individual and interactions amongst the nodes.

Next at block 236, the parameter optimizer 164 (e.g., GNN) may be applied to the matrix to optimize the binary action (e.g., no change or change (e.g., predefined increase or decrease)) to generate a candidate modulator configuration. In some examples, the graphical representation may be updated based on the optimized binary actions (e.g., maintain, increase, or decrease width of a component within each pin). By way of example, the matrix may also be simultaneously updated with a new set of elements to reflect these changes, ensuring that the objective function remains accurately aligned with the modified modulator configuration. In some examples, the updated matrix may be fed back into the GNN as part of the backpropagation process, allowing for the refinement of the network's internal parameters in preparation for subsequent iterations.

Next, at block 238, the candidate modulator configuration may be evaluated to determine whether it satisfies stopping criteria. In some examples, one or more the cost values of the candidate modulator configuration may be determined using the respective cost function(s) and compared to the respective plan objective(s). If the cost value does not meet the criteria for the plan (NO at block 238) or other stopping criteria is not met (e.g., maximum number of iterations), another iteration of the first optimization procedure 230 may be performed. At block 232, the graphical representation from block 236 may be updated by re-initializing the binary actions to zero. In some examples, the second iteration may have a different predefined action associated with binary variable “1”. In some examples, the first optimization procedure 230 (blocks 232-238) may be repeated until the stopping criteria are satisfied at block 238.

If the cost value meets the criteria for plan objective (e.g., cost value is within specific range and/or maximum number of iterations (blocks 232-238) of the first optimization procedure 230 have been performed) (YES at block 238), the operations may optionally continue to the second optimization procedure 250 related to optimizing the spot intensity.

At block 252, a graphical representation for spot intensity may be generated by updating the graphical representation from block 254. In this graphical representation, each node may represent a proton spot with binary action and physical features(s). The graphical representation may be updated to initiate the action for each node to 0. In some examples, the weight of each spot may be directly related to the intensity of radiation delivered to the target volume within the patient.

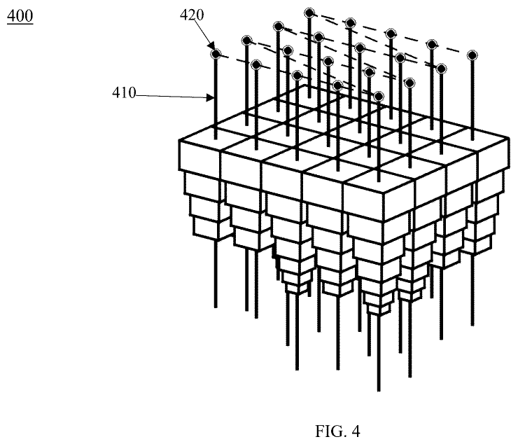

FIG. 4 shows an illustrative example 400 of a graphical representation generated for spot intensity. In this example, each monoenergetic proton beam (line 410) passes through its unit of modulator and the spots (black dot 420) can be characterized by its position and Monitor Units (MU) (intensity).

Next, at block 254, the matrix generator 162 may generate a matrix for the spot intensity using the graphical representation and the optimization objectives derived from the clinical goals.

By way of example, for spot intensity, a matrix, denoted as Qᵢⱼ, may be constructed such that each element of this matrix may represent the change in the quantitative measure Δf corresponding to a predefined percentage change in the intensity of individual spots or pairs of spots.

$${Q}_{i j} = {\sum }_{i} Δ {f}_{i} {x}_{i} + {\sum }_{i < j} Δ {f}_{i j} {x}_{i} {x}_{j}$$

Δfᵢ and Δfᵢⱼ are the diagonal and nondiagonal elements of matrix Qᵢⱼ. Each proton spot can be represented by the binary variable xᵢ.

In some examples, the predefined percentage change may be based on the iteration number of the optimization procedure. By way of example, the predefined change for the first iteration of the (first) optimization procedure may be increase the spot intensity by a predefined percentage (e.g., 10%) (e.g., associated with binary value 1). The predefined change may differ between iterations.

Next at block 256, the parameter optimizer 164 (e.g., GNN) may be applied to the matrix to optimize the binary action (e.g., no change or change (e.g., predefined increase or decrease)) to generate a therapy parameter (e.g., spot intensity) candidate. In some examples, the graphical representation may be updated based on the optimized binary actions (e.g., maintain, increase, or decrease width of a spot intensity within each pin) determined by the GNN.

By way of example, the matrix (Qᵢⱼ) may also be simultaneously updated with a new set of elements to reflect these changes, ensuring that the objective function remains accurately aligned with the modified spot intensities. In some examples, the updated matrix (Qᵢⱼ) may be fed back into the GNN as part of the backpropagation process, allowing for the refinement of the network's internal parameters in preparation for subsequent iterations.

Next, at block 258, the candidate therapy parameter(s) may be evaluated to determine whether it satisfies stopping criteria. In some examples, one or more the cost values of the candidate therapy parameter(s) may be determined using the respective cost function(s) and compared to the respective plan objective(s). If the cost value does not meet the criteria for the plan (NO at block 258) or other stopping criteria is not met (e.g., maximum number of iterations), another iteration of the second optimization procedure 250 may be performed. At block 252, the graphical representation from block 256 may be updated by re-initializing the binary actions to zero. In some examples, the second iteration may have a different predefined action associated with binary variable “1”. For example, the predefined action associated with binary variable “1” may include X. In some examples, the second optimization procedure 250 (blocks 252-258) may be repeated until the stopping criteria are satisfied at block 258.

If the cost value meets the criteria for plan objective (e.g., cost value is within specific range and/or maximum number of iterations (blocks 252-258) of the second procedure 258 have been performed) (YES at block 258), the operations may optionally continue to block 260.

At block 260, the candidate treatment plan generated using the candidate modulator configuration and candidate spot intensity that satisfies the respective stopping criteria (YES at blocks 238 and 258) may be evaluated to determine whether the candidate treatment plan satisfies stopping criteria. In some examples, one or more the cost values of the candidate treatment plan may be determined using the respective cost function(s) and compared to the respective plan objective(s). If the cost value does not meet the criteria for the plan (NO at block 260) or other stopping criteria is not met (e.g., maximum number of iterations), another iteration of the optimization procedures (blocks 230 and 250) may be performed. In this example, the binary action associated with modulator configuration may be reinitialized to “O” before operations are performed at block 220.

If each cost value meets the criteria for respective plan objective (e.g., each cost value is within specific range and/or maximum number of iterations of the optimization procedures 230 and 250 have been performed) (YES at block 260), the operations continue to block 270.

At block 270, the final treatment plan may be outputted. For example, the final treatment plan may be transmitted, stored, and/or displayed. In some examples, the method 200 may transmit the beam modulator configuration to the beam modulator construction device 170 that can construct the beam modulator device 172 using the corresponding modular components. For example, a robotic system may construct/build the beam modulator configuration using the pre-fabricated modular components. In some examples, the method may further include performing quality control, using optical imaging.

In some examples, at block 270, the method 200 may transmit the therapy parameters 142 to the radiation therapy system 180. This way, the radiation therapy system 180 may deliver radiation therapy (e.g., proton therapy) to a patient according to the final treatment plan using the beam modulator device 172 fabricated according to the final treatment plan.

In some examples, the first optimization procedure 230 and the second optimization procedure 250 may be performed sequentially as shown in FIG. 2. In some examples, the first optimization procedure 230 and the second optimization procedure 250 may be performed simultaneously to optimize both the beam modulator configuration 144 and the therapy parameter(s) 142. For example, the optimization module 160 may be configured to optimize the modulator configuration 144 and the therapy parameter(s) 142 simultaneously. By way of example, the graphical representation may include nodes representing both parameters 142 and 146, the matrix generator 162 may be configured to generate a matrix incorporating all elements, the parameter optimizer 164 may be configured to optimize variables for both parameters 142 and 146, and the cost function 166 may relate to both parameters 142 and 144.

In some examples, a different binary action may be optimized. For example, the first and/or second optimization procedures may be optimized to select the specific geometric component for the modulator configuration 144 and/or specific intensity value for each spot for the therapy parameter(s) 146, for example, by using one-hot encoding. In this example, the binary value of “1” may indicate “selection” of that specific size/intensity value at the specific unit and/or spot and binary value of “0” may indicate “no selection” at the specific unit and/or spot.

By way of example, for the modulator configuration 144, the graphical representation may be generated so that each node can represent the choice of a specific size of the geometric component at a specific unit position. In this example, the iterative optimization procedure may be performed to determine the optimal combination of components across all positions. In some examples, the optimal combination of component sizes from a predefined library (e.g., [1 mm, 2 mm, 3 mm, 4 mm, 5 mm]) across all pin positions (e.g., 3-6 layers per unit) may be considered the combination that results in maximization of the overall treatment plan quality (i.e., cost function is minimized):

In this example, the graphical representation may include additional information. For example, the graphical representation (of nodes corresponding to the (k) number of position(s)) may be modified to also include a node for each position and step size (n). By way of example, for k step positions and n step size options, the graphical representation may include k×n binary nodes (e.g., for 4 positions and 5 step sizes→20 nodes).

In this example, each node's binary variable x_{i,j} can represent: x_{i,j}=1→choose geometric component size sⱼ at position I; and x_{i,j}=0→not selected.

In this example, the initial configuration may be generated with a random and/or uniform geometric component size. The cost value may be determined for the initial configuration. The matrix may be generated using the graphical representation and cost value. In some examples, constraint penalties may be added to the matrix to enforce one-hot encoding per position.

In some examples, the parameter optimizer 164 (e.g., quantum annealing, simulated annealing, or a GNN-based solver) may be applied to the matrix to find a new set of binary values that minimize the cost function.

The node values may be updated. For example, binary activations may be updated based on the optimization results and the modulator configuration may be updated based on the activations. The updated modulator configuration may be stopping criteria. If stopping criteria is not satisfied, another iteration of the optimization procedure may be performed with different step sizes.

This process can be similarly performed for the second optimization procedure by replacing the random or specific sized geometric components with random or specific spot intensities.

In some examples, the first optimization procedure 230 and/or the second optimization procedure 250 may be omitted. For example, the first optimization procedure 230 (steps 210-238 and 270) may be performed so that only the modulator configuration 144 is optimized. By way of another example, the second optimization procedure 250 (steps 210, 252-258, and 270) may be performed so that only the treatment parameters 142 (e.g., spot intensity) may be optimized.

FIG. 5 shows an example 500 of workflow to optimize only the modulator configuration 144. In this example, the initial modulator configuration 310 may generated based on an initial treatment plan using the geometric components (M_{I}-M_{IV}) 552 stored in the library 150. In this example, the initial modulator configuration 310 may include the units 320 and 330. The unit 320 may include three components n₁-n₃ stacked and the unit 330 may include three components n₄-n₆ stacked. In this example, the initial configuration of the unit 320 may include components M_{I}-M_{III} in positions n₁-n₃, respectively; and the initial configuration of the unit 330 may include components M_{I}, M_{III}, and M_{IV} in positions n₃-n₆.

Next, a graphical representation 340 of the modulator configuration may be generated from the initial beam modulator configuration 310. As shown, the graphical representation 340 may include nodes representing individual components n₁-n₆ and their associated features (e.g., including features of respective components (M_{I}-M_{IV})). In the first iteration, the binary actions in the graphical representation associated with each node may be initialized to 0.

Next, a matrix 562 (corresponding to matrix 350) that include elements representing a predefined change in the component size of the respective component of the modulator configuration 310 may be generated using the graphical representation 340 and the optimization objectives derived from the clinical goals. In some examples, the predefined change may be based on the iteration number of the optimization procedure. By way of example, the predefined change for the first iteration of the (first) optimization procedure may be increase the width of a component (e.g., associated with binary value 1) and/or the height of a component (e.g., associated with binary value 1) of the configuration.

Next, a GNN 564 may be applied to the matrix 562 to optimize the binary action (e.g., no change or change (e.g., predefined increase or decrease)) to generate a candidate modulator configuration. The GNN may generate the optimized binary actions 566. As shown in this example, the binary values for nodes n′₁ and n′₆ may indicate a predefined change. In this example, “1” indicates a predefined incremental increase in component size. The modular configuration may be updated according to the optimized binary actions 566.

In some examples, the graphical representation 340 may be updated (arrow 520) based on the optimized binary actions 566 (e.g., maintain, increase, or decrease width of a component within each pin). By way of example, the matrix 562 may also be simultaneously updated with a new set of binary values to reflect these changes, ensuring that the objective function remains accurately aligned with the modified modulator configuration. In some examples, the updated matrix 562 may be fed back into the GNN as part of the backpropagation process (arrow 510), allowing for the refinement of the network's internal parameters in preparation for subsequent iterations.

In this example, the configuration associated with binary actions 566 satisfy the stopping criteria so the optimization procedure can terminate. Next, the final treatment plan using the final configuration may be transmitted to the beam modulator construction device 170 for construction. In this example, the beam modulator construction device 170 may be a robotic arm that fabricates the beam modulator device 572 using pre-fabricated modular components corresponding to the components (M_{I}-M_{IV}) stored in the library 552. As shown in FIG. 5, the built modulator device 572, corresponding to the final modulator configuration, includes components M_{I}, M_{III}, and M_{IV} in positions n₁-n₃ for unit 320, respectively; and components M_{II}, M_{III}, and M_{IV} in positions n₄-n₆ for unit 330. As shown, the final modulator configuration includes components for n₁ and ne that are larger than the initial configuration 310. In this example, the component n₂ has been changed from M_{II} to M_{III} and the component n₄ has been changed from M_{I} to M_{II}.

FIG. 6 depicts a block diagram of an example computing system 600 for implementing certain embodiments. For example, in some aspects, the computer system 600 may include computing systems associated with device(s) performing one or more processes (e.g., FIGS. 2 and 5) disclosed herein. The block diagram illustrates some electronic components or subsystems of the computing system. The computing system 600 depicted in FIG. 6 is merely an example and is not intended to unduly limit the scope of inventive embodiments recited in the claims. One of ordinary skill in the art would recognize many possible variations, alternatives, and modifications. For example, in some implementations, the computing system 600 may have more or fewer subsystems than those shown in FIG. 6, may combine two or more subsystems, or may have a different configuration or arrangement of subsystems.

In the example shown in FIG. 6, the computing system 600 may include one or more processing units 610 and storage 620. The processing units 610 may be configured to execute instructions for performing various operations, and can include, for example, any combination of micro-controllers, general-purpose processors, dedicated processors (e.g., graphics processor (GPU), application processors, etc.) and/or a microprocessor suitable for implementation within a portable electronic device, such as a Raspberry Pi. The processing units 610 may be communicatively coupled with a plurality of components within the computing system 600. For example, the processing units 610 may communicate with other components across a bus. The bus may be any subsystem adapted to transfer data within the computing system 600. The bus may include a plurality of computer buses and additional circuitry to transfer data.

In some embodiments, the processing units 610 may be coupled to the storage 620. In some embodiments, the storage 620 may offer both short-term and long-term storage and may be divided into several units. The storage 620 may be volatile, such as static random access memory (SRAM) and/or dynamic random access memory (DRAM), and/or non-volatile, such as read-only memory (ROM), flash memory, and the like. Furthermore, the storage 620 may include removable storage devices, such as secure digital (SD) cards. The storage 620 may provide storage of computer readable instructions, data structures, program modules, audio recordings, image files, video recordings, and other data for the computing system 600. In some embodiments, the storage 620 may be distributed into different hardware modules. A set of instructions and/or code might be stored on the storage 620. The instructions might take the form of executable code that may be executable by the computing system 600, and/or might take the form of source and/or installable code, which, upon compilation and/or installation on the computing system 600 (e.g., using any of a variety of generally available compilers, installation programs, compression/decompression utilities, and the like), may take the form of executable code.

In some embodiments, the storage 620 may store a plurality of application modules 624, which may include any number of applications, such as applications for controlling input/output (I/O) devices 640 (e.g., a sensor, a switch, a camera, a microphone or audio recorder, a speaker, a media player, a display device, etc.). The application modules 624 may include particular instructions to be executed by the processing units 610. In some embodiments, certain applications or parts of the application modules 624 may be executable by other hardware modules, such as a communication subsystem 650. In certain embodiments, the storage 620 may additionally include secure memory, which may include additional security controls to prevent copying or other unauthorized access to secure information.

In some embodiments, the storage 620 may include an operating system 622 loaded therein, such as an Android operating system or any other operating system suitable for mobile devices or portable devices. The operating system 622 may be operable to initiate the execution of the instructions provided by the application modules 624 and/or manage other hardware modules as well as interfaces with a communication subsystem 650 which may include one or more wireless or wired transceivers. The operating system 622 may be adapted to perform other operations across the components of the computing system 600 including threading, resource management, data storage control, and other similar functionality.

The communication subsystem 650 may include, for example, an infrared communication device, a wireless communication device and/or chipset (such as a Bluetooth® device, an IEEE 802.11 (Wi-Fi) device, a WiMax device, cellular communication facilities, and the like), NFC, ZigBee, and/or similar communication interfaces. The computing system 600 may include one or more antennas (not shown in FIG. 6) for wireless communication as part of the communication subsystem 650 or as a separate component coupled to any portion of the system.

Depending on desired functionality, the communication subsystem 650 may include separate transceivers to communicate with base transceiver stations and other wireless devices and access points, which may include communicating with different data networks and/or network types, such as wireless wide-area networks (WWANs), WLANs, or wireless personal area networks (WPANs). A WWAN may be, for example, a WiMax (IEEE 802.9) network. A WLAN may be, for example, an IEEE 802.11x network. A WPAN may be, for example, a Bluetooth network, an IEEE 802.15x, or some other types of network. The techniques described herein may also be used for any combination of WWAN, WLAN, and/or WPAN. In some embodiments, the communications subsystem 650 may include wired communication devices, such as Universal Serial Bus (USB) devices, Universal Asynchronous Receiver/Transmitter (UART) devices, Ethernet devices, and the like. The communications subsystem 650 may permit data to be exchanged with a network, other computing systems, and/or any other devices described herein. The communication subsystem 650 may include a means for transmitting or receiving data, such as identifiers of portable goal tracking devices, position data, a geographic map, a heat map, photos, or videos, using antennas and wireless links. The communication subsystem 650, the processing units 610, and the storage 620 may together comprise at least a part of one or more of a means for performing some functions disclosed herein.

The computing system 600 may include one or more I/O devices 640, such as a switch, a camera, a microphone or audio recorder, a communication port, or the like. For example, the I/O devices 640 may include one or more touch sensors or button sensors associated with the buttons. The touch sensors or button sensors may include, for example, a mechanical switch or a capacitive sensor that can sense the touching or pressing of a button.

In some embodiments, the I/O devices 640 may include a microphone or audio recorder that may be used to record an audio message. The microphone and audio recorder may include, for example, a condenser or capacitive microphone using silicon diaphragms, a piezoelectric acoustic sensor, or an electret microphone. In some embodiments, the microphone and audio recorder may be a voice-activated device. In some embodiments, the microphone and audio recorder may record an audio clip in a digital format, such as MP6, WAV, WMA, DSS, etc. The recorded audio files may be saved to the storage 620 or may be sent to the one or more network servers through the communication subsystem 650.

In some embodiments, the I/O devices 640 may include a location tracking device, such as a global positioning system (GPS) receiver. In some embodiments, the I/O devices 640 may include a wired communication port, such as a micro-USB, Lightning, or Thunderbolt transceiver.

The I/O devices 640 may also include, for example, a speaker, a media player, a display device, a communication port, or the like. For example, the I/O devices 640 may include a display device, such as an LED or LCD display and the corresponding driver circuit. The I/O devices 640 may include a text, audio, or video player that may display a text message, play an audio clip, or display a video clip.

The computing system 600 may include a power device 640, such as a rechargeable battery for providing electrical power to other circuits on the computing system 600. The rechargeable battery may include, for example, one or more alkaline batteries, lead-acid batteries, lithium-ion batteries, zinc-carbon batteries, and NiCd or NiMH batteries. The computing system 600 may also include a battery charger for charging the rechargeable battery. In some embodiments, the battery charger may include a wireless charging antenna that may support, for example, one of Qi, Power Matters Association (PMA), or Association for Wireless Power (A6WP) standard, and may operate at different frequencies. In some embodiments, the battery charger may include a hard-wired connector, such as, for example, a micro-USB or Lightning® connector, for charging the rechargeable battery using a hard-wired connection. The power device 640 may also include some power management integrated circuits, power regulators, power converters, and the like.

The computing system 600 may be implemented in many different ways. In some embodiments, the different components of the computing system 600 described above may be integrated to a same printed circuit board. In some embodiments, the different components of the computing system 600 described above may be placed in different physical locations and interconnected by, for example, electrical wires. The computing system 600 may be implemented in various physical forms and may have various external appearances. The components of computing system 600 may be positioned based on the specific physical form.

The methods, systems, and devices discussed above are examples. Various embodiments may omit, substitute, or add various procedures or components as appropriate. For instance, in alternative configurations, the methods described may be performed in an order different from that described, and/or various stages may be added, omitted, and/or combined. Also, features described with respect to certain embodiments may be combined in various other embodiments. Different aspects and elements of the embodiments may be combined in a similar manner. Also, technology evolves and, thus, many of the elements are examples that do not limit the scope of the disclosure to those specific examples.

The foregoing method descriptions and the process flow diagrams are provided merely as illustrative examples and are not intended to require or imply that the operations of various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an” or “the” is not to be construed as limiting the element to the singular.

While the terms “first” and “second” are used herein to describe data transmission associated with a subscription and data receiving associated with a different subscription, such identifiers are merely for convenience and are not meant to limit various embodiments to a particular order, sequence, type of network or carrier.

Various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such embodiment decisions should not be interpreted as causing a departure from the scope of the claims.

The hardware used to implement various illustrative logics, logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing systems, (e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In one or more example embodiments, the functions described may be implemented in hardware, software, firmware, or any combination thereof. If implemented in software, the functions may be stored as one or more instructions or code on a non-transitory computer readable medium or non-transitory processor-readable medium. The operations of a method or algorithm disclosed herein may be embodied in a processor-executable software module, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable medium and/or computer-readable medium, which may be incorporated into a computer program product.

Those of skill in the art will appreciate that information and signals used to communicate the messages described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Terms, “and” and “or” as used herein, may include a variety of meanings that also is expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AC, BC, AA, ABC, AAB, AABBCCC, and the like.

Further, while certain embodiments have been described using a particular combination of hardware and software, it should be recognized that other combinations of hardware and software are also possible. Certain embodiments may be implemented only in hardware, or only in software, or using combinations thereof. In one example, software may be implemented with a computer program product containing computer program code or instructions executable by one or more processors for performing any or all of the steps, operations, or processes described in this disclosure, where the computer program may be stored on a non-transitory computer readable medium. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques, including, but not limited to, conventional techniques for inter-process communications, and different pairs of processes may use different techniques, or the same pair of processes may use different techniques at different times.

The disclosures of each and every publication cited herein and cited in the Appendices are hereby incorporated herein by reference in their entirety.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

What is claimed:

1. A method, comprising:
applying one or more optimization procedures to one or more candidate radiation treatment plans for radiation treatment in a patient according to one or more plan optimization objectives associated with one or more cost functions to generate a final radiation treatment plan;
wherein:
each radiation treatment plan includes therapy parameters and a beam modulator configuration of one or more geometric components from a library storing a plurality of the modular components;
one or more optimization procedures is applied to the beam modulator configuration of each candidate radiation treatment plan to generate a final beam modulator configuration; and
the final treatment plan including the final beam modulator configuration.

2. The method according to claim 1, further comprising:
fabricating a beam modulator using the final beam modulator configuration and one or more prefabricated beam modulator components corresponding to the one or more geometric components stored in the library.

3. The method according to claim 1, wherein the one or more optimization procedures includes one or more iterations, each iteration optimizing a predefined binary action associated with one or more parameters.

4. The method according to claim 1, wherein:
the one or more optimization procedures includes one or more optimization procedures that are applied to the one or more of the therapy parameters of each candidate radiation treatment plan to generate one or more final therapy parameters;
the one or more of the therapy parameters including spot intensity; and
the final treatment plan including the final therapy parameters.

5. The method according to claim 1, wherein each optimization procedure includes:
generating a graphical representation of one or more candidate parameters of the candidate treatment plan to be optimized;
wherein each node of the graphical representation represents a binary action associated with one or more parameters; and
wherein the one or more candidate parameters includes the one or more of the therapy parameters and/or the beam modulator configuration.

6. The method according to claim 5, wherein:
the binary action associated with beam modulator configuration includes a binary value representing an action with respect to the respective geometric component; and
the binary value including (i) a value representing a predefined increase or decrease in size of respective geometric component of the beam modulator configuration; and (ii) a value representing a maintaining of a size of the respective geometric component.

7. The method according to claim 5, wherein:
the binary action associated with therapy parameter configuration includes a binary value representing an action with respect to respective spot intensity; and
the binary value including (i) a value representing a predefined increase or decrease in respective spot intensity of the beam modulator configuration; and (ii) a value representing a maintaining of a size of the respective spot intensity.

8. The method according to claim 5, wherein each optimization procedure further includes:
generating a matrix that includes elements having a value representing a predefined change in the candidate parameter using the graphical representation and the optimization objectives derived from the clinical goals.

9. The method according to claim 8, wherein each optimization procedure includes:
applying a graphical neural network to the matrix to evaluate combinations of binary variables associated with a binary action to minimize a cost value to determine the binary action for each candidate parameter.

10. A system, comprising:
one or more processors; and
one or more hardware storage devices having stored thereon computer-executable instructions which are executable by the one or more processors to cause the computing system to perform at least the following:
applying one or more optimization procedures to one or more candidate radiation treatment plans for radiation treatment in a patient according to one or more plan optimization objectives associated with one or more cost functions to generate a final radiation treatment plan;
wherein:
each radiation treatment plan includes therapy parameters and a beam modulator configuration of one or more geometric components from a library storing a plurality of the modular components;
one or more optimization procedures is applied to the beam modulator configuration of each candidate radiation treatment plan to generate a final beam modulator configuration; and
the final treatment plan including the final beam modulator configuration.

11. The system according to claim 10, wherein the one or more processors are further configured to cause the computing system to perform at least the following:
fabricating a beam modulator using the final beam modulator configuration and one or more prefabricated beam modulator components corresponding to the one or more geometric components stored in the library.

12. The system according to claim 10, wherein the one or more optimization procedures includes one or more iterations, each iteration optimizing a predefined binary action associated with one or more parameters.

13. The system according to claim 10, wherein:
the one or more optimization procedures includes one or more optimization procedures that are applied to the one or more of the therapy parameters of each candidate radiation treatment plan to generate one or more final therapy parameters;
the one or more of the therapy parameters including spot intensity; and
the final treatment plan including the final therapy parameters.

14. The system according to claim 10, wherein each optimization procedure includes:
generating a graphical representation of one or more candidate parameters of the candidate treatment plan to be optimized;
wherein each node of the graphical representation represents a binary action associated with one or more parameters; and
wherein the one or more candidate parameters includes the one or more of the therapy parameters and/or the beam modulator configuration.

15. The system according to claim 14, wherein:
the binary action associated with beam modulator configuration includes a binary value representing an action with respect to the respective geometric component; and
the binary value including (i) a value representing a predefined increase or decrease in size of respective geometric component of the beam modulator configuration; and (ii) a value representing a maintaining of a size of the respective geometric component.

16. The system according to claim 14, wherein:
the binary action associated with therapy parameter configuration includes a binary value representing an action with respect to respective spot intensity; and
the binary value including (i) a value representing a predefined increase or decrease in respective spot intensity of the beam modulator configuration; and (ii) a value representing a maintaining of a size of the respective spot intensity.

17. The system according to claim 14, wherein each optimization procedure further includes:
generating a matrix that includes elements having a value representing a predefined change in the candidate parameter using the graphical representation and the optimization objectives derived from the clinical goals.

18. The system according to claim 17, wherein each optimization procedure includes:
applying a graphical neural network to the matrix to evaluate combinations of binary variables associated with a binary action to minimize a cost value to determine the binary action for each candidate parameter.
